(19) United States
(12) Patent Application Publication
(10) Pub. No.: US 2016/0183834 A1
(43) Pub. Date: Jun. 30, 2016

(54) SEMI-CONTACT-TYPE ECG MEASUREMENT SYSTEM AND MEASUREMENT METHOD THEREOF

(71) Applicants: HYUNDAI MOTOR COMPANY, Seoul (KR); KIA MOTORS CORPORATION, Seoul (KR); TYCO ELECTRONICS AMP KOREA CO., LTD., Gyeongsan-si (KR); HYUNDAI DYMOS INC., Seosan-si (KR)

(72) Inventors: Byung Jae LEE, Gunpo-si (KR); Yong Soo CHANG, Seoul (KR); Kyu Ho LEE, Seoul (KR); Hyun Seok SONG, Yeosu-si (KR); Kwang Ho SONG, Incheon (KR); Young Deok KIM, Seoul (KR); Tae Hyoung YANG, Seoul (KR); In Ho LEE, Hwaseong-si (KR); Taek Jun NAM, Seongnam-si (KR)

(21) Appl. No.: 14/939,655

(22) Filed: Nov. 12, 2015

(30) Foreign Application Priority Data
Dec. 31, 2014 (KR) 10-2014-0195499

(51) Int. Cl. A61B 5/0456; A61B 5/00; A61B 5/0245; A61B 5/04

(52) U.S. Cl. CPC A61B 5/0456; A61B 5/04017; A61B 5/7203; A61B 5/0245; A61B 5/6893

(57) ABSTRACT

The present disclosure provides a semi-contact type ECG measurement system, comprising a contact-type sensor configured to directly contact a skin of a passenger in a vehicle and detect a first ECG signal; a non-contact type sensor configured to be in proximity to the passenger to detect a second ECG signal without directly contacting the skin of the passenger; and an ECG sensor module configured to process the first and second ECG signals and configured to determine a biological status of the passenger from the first and second ECG signals microcontroller.

### CROSS-REFERENCE TO RELATED APPLICATION

This application claims under 35 U.S.C. §119(a) the benefit of priority to Korean Patent Application No. 10-2014-0195499, filed on Dec. 31, 2014, the entire content of which is incorporated herein by reference.

### TECHNICAL FIELD

The present disclosure relates to an ECG measurement system. More particularly, it relates to a semi-contact-type ECG measurement system and measurement method thereof having excellent convenience for nonrestraint measurement as compared with an existing contact-type heart-rate detection method and also having excellent ECG signal quality as compared with a non-contact type heart-rate detection method.

### BACKGROUND

In the medical field, an electrocardiogram (ECG) measurement device is a widely-used medical instrument conventionally used in a manner of contacting an electrode to measure a body's potential and detecting electrical activity and recording the detected electrical activity in the form of a graph.

In recent years, there has been ongoing research into a technique that measures the ECG of a vehicle driver while the driver is driving the vehicle.

One of the primary reasons for the research activity is to monitor the heart activity of the driver to resolve various inconveniences caused by a heart problem that may occur while the driver is driving and to prevent an accident which may occur in the event of cardiac arrest.

The above information disclosed in this Background section is only for the enhancement of understanding of the background of the inventive concept and therefore it may contain information that does not form the prior art that is already known in this country to a person of ordinary skill in the art.

### SUMMARY

The present disclosure has been made in an effort to solve the above-described problems associated with prior art.

The present disclosure has been made in an effort to provide a semi-contact-type ECG measurement system and a measurement method thereof having excellent convenience by nonrestraint measurement as compared with an existing contact-type heart-rate detection method and excellent ECG signal quality as compared with a non-contact type heart-rate detection method, by directly contacting a first electrode and a GND on the skin and configuring a second electrode by a two-point contact method in which the second electrode contacts the skin with clothes therebetween by a non-contact type in order to better detect ECG signals.

In one aspect, the present inventive concept provides a semi-contact type ECG measurement system including: a contact-type sensor positioned at a portion directly contacting a skin of a passenger in a vehicle to contact the exposed skin of the passenger and detect an ECG signal; a non-contact type sensor positioned in close contact with the clothes of the passenger at a seat to detect the ECG signal through the clothing of the passenger; and an ECG sensor module transmitting and processing the ECG signal to provide a stable environment in a vehicle by determining a biological status of the passenger by the ECG signal detected by the contact-type sensor and the non-contact type sensor.

In another aspect, the present inventive concept provides a semi-contact type ECG measurement method including: measuring an ECG by detecting an ECG signal of a passenger seated on a seat through a semi-contact type ECG measurement system; storing and collecting ECG data measured at multiple predetermined times; obtaining ECG R-R PEAK INTERVAL information in the ECG data collected through filtering; determining whether the obtained ECG R-R PEAK INTERVAL information has regularity; calculating a stress index by analyzing and implementing a stress algorithm in the ECG R-R PEAK INTERVAL information through a stress index calculating unit; and creating a measurement and an environment for maintaining the stability of the passenger through the Microcontroller based on the calculated stress index.

According to the present inventive concept configured above, the present disclosure provides a semi-contact type ECG measurement system, comprising a contact-type sensor configured to directly contact a skin of a passenger in a vehicle and detect a first ECG signal; a non-contact type sensor configured to be in proximity to the passenger to detect a second ECG signal without directly contacting the skin of the passenger; and an ECG sensor module configured to process the first and second ECG signals and configured to determine a biological status of the passenger from the first and second ECG signals.

The ECG sensor module may further comprise a differential amplifying unit configured to amplify the first and second ECG signals and to remove common mode noise; a band pass filter configured to remove noise; an A/D converter; an R-R PEAK detector configured to detect an R-R PEAK INTERVAL; and a stress index calculator configured to calculate a stress index based on the R-R PEAK INTERVAL.

The ECG sensor module may further comprise a notch filter configured to remove a power noise component from the ECG signal.

A semi-contact type ECG measurement method may comprise steps of measuring an ECG by detecting an ECG signal of a passenger seated on a seat through a semi-contact type ECG measurement system; collecting ECG data measured at predetermined times; obtaining ECG R-R PEAK INTERVAL information in the ECG data; determining whether the obtained ECG R-R PEAK INTERVAL information has regularity; calculating a stress index using the ECG R-R PEAK INTERVAL information; and outputting a measurement based on the stress index.

The semi-contact type ECG measurement method may further comprise initializing an ECG signal and a PEAK value before the ECG measurement step.

In addition, a body reaction index such as s heart-rate, an ECG, and stress may be calculated without disturbing the driver, and a safe driving environment in a vehicle may be provided by a feedback of a MICROCONTROLLER corresponding to the calculated result.

Other aspects and exemplary embodiments of the inventive concept are discussed infra.

It is understood that the term “vehicle” or “vehicular” or other similar term as used herein is inclusive of motor vehicles in general such as passenger automobiles including sports utility vehicles (SUV), buses, trucks, various commercial vehicles, watercraft including a variety of boats and ships, aircraft, and the like, and includes hybrid vehicles, electric vehicles, plug-in hybrid electric vehicles, hydrogen-powered vehicles and other alternative fuel vehicles (e.g. fuels derived from resources other than petroleum). As referred to herein, a hybrid vehicle is a vehicle that has two or more sources of power, for example both gasoline-powered and electric-powered vehicles.

### BRIEF DESCRIPTION OF THE DRAWINGS

The above and other features of the present inventive concept will now be described in detail with reference to certain exemplary embodiments thereof illustrated the accompanying drawings which are given hereinbelow by way of illustration only, and thus are not limitative of the present inventive concept, and wherein:

FIG. 1 is a configuration diagram of a semi-contact type ECG measurement system according to an embodiment of the present inventive concept.

FIG. 2—is a configuration diagram of an ECG sensor module in the semi-contact type ECG measurement system according to an embodiment of the present inventive concept.

FIGS. 3 and 4 are configuration and installation state diagrams of the semi-contact type ECG measurement system according to an embodiment of the present inventive concept which is applied in a vehicle.

FIG. 5 is a flowchart for an ECG measurement method through the semi-contact type ECG measurement system according to an embodiment of the present inventive concept.

It should be understood that the appended drawings are not necessarily to scale, presenting a somewhat simplified representation of various features illustrative of the basic principles of the inventive concept. The specific design features of the present inventive concept as disclosed herein, including, for example, specific dimensions, orientations, locations, and shapes will be determined in part by the particular intended application and use environment.

In the figures, reference numbers refer to the same or equivalent parts of the present inventive concept throughout the several figures of the drawing.

### DETAILED DESCRIPTION

Hereinafter reference will now be made in detail to various embodiments of the present inventive concept, examples of which are illustrated in the accompanying drawings and described below. While the inventive concept will be described in conjunction with exemplary embodiments, it will be understood that present description is not intended to limit the inventive concept to those exemplary embodiments. On the contrary, the inventive concept is intended to cover not only the exemplary embodiments, but also various alternatives, modifications, equivalents and other embodiments, which may be included within the spirit and scope of the inventive concept as defined by the appended claims.

The present inventive concept will be described more fully hereinafter with reference to the accompanying drawings, in which exemplary embodiments of the inventive concept are shown.

Before describing the present inventive concept, an electrocardiogram (ECG) is a device used to measure a heart rate as a kind of waveform by inducing, differentiating, and amplifying a potential change generated by activity of heart muscles (depolarization/repolarization) by an electrode 130 in contact with a body surface.

That is, a sensor in which two electrodes 130 (one is a capacitive-coupling electrode and the other one is an electrode for measuring a potential difference) and a GND 131 are separated from each other and integrally formed is provided on a vehicle seat 100 that may contact a human body, and another sensor is provided on the vehicle seat 100 that may not contact the human body.

Here, the contact-type sensor measures a signal generated by a difference between a potential of human skin contacting the electrodes 130 and a potential of human skin contacting the GND 131 surrounding the electrodes 130, and the electrode 130 of the non-contact type sensor measures a signal generated by a difference between a potential generated by an electric field flowing from the human body through covering by the capacitive-coupling electrode 131 and a potential of the human body contacting the GND 131 of the contact type sensor unit connected with a circuit. Accordingly, the ECG signal is measured as a difference between the signal measured in the contact-type sensor 110 and the signal measured in the non-contact type sensor 120.

In this case, the configuration of each sensor unit provides the electrode 130 and the GND 131, the electrode 130 part of a portion to be measured is influenced by the electric field flowing through the contact or non-contact human body, and the electric field is influenced by the GND 131 formed around the electrode 130.

Accordingly, a direction and a size of the electric field flowing into the electrode 130 may be configured to be similar to a size of external noise by the GND 131, and as a result, a structure in which noise is easily removed by a difference between two signals measured by two sensors and the ECG may be obtained, and a signal may be stably obtained as compared with the non-contact type sensor based on excellent signal quality of the contact-type sensor.

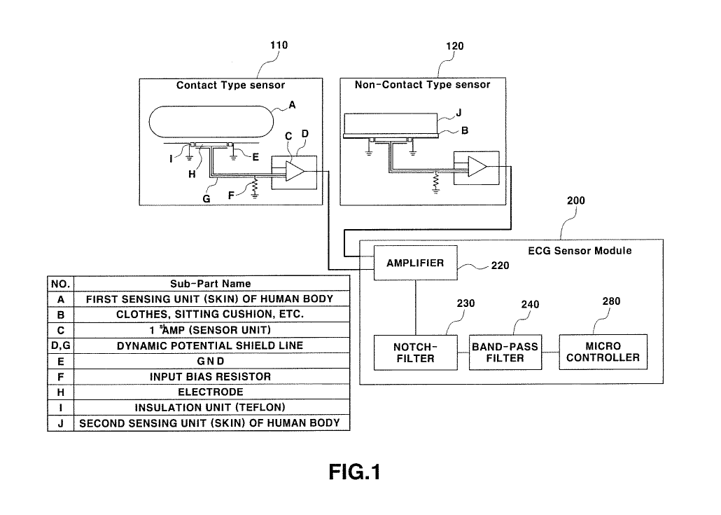

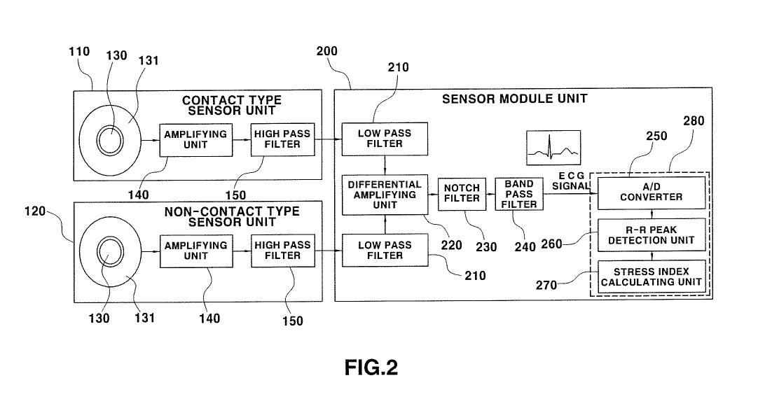

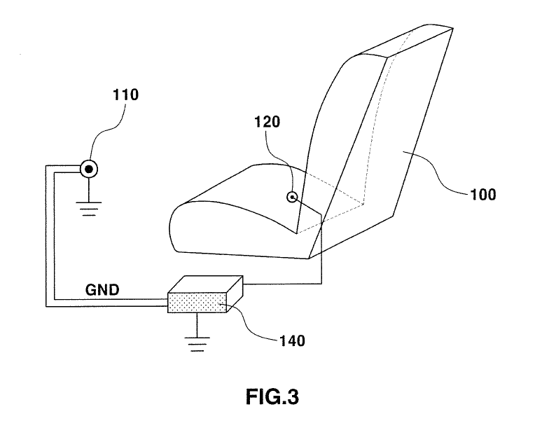

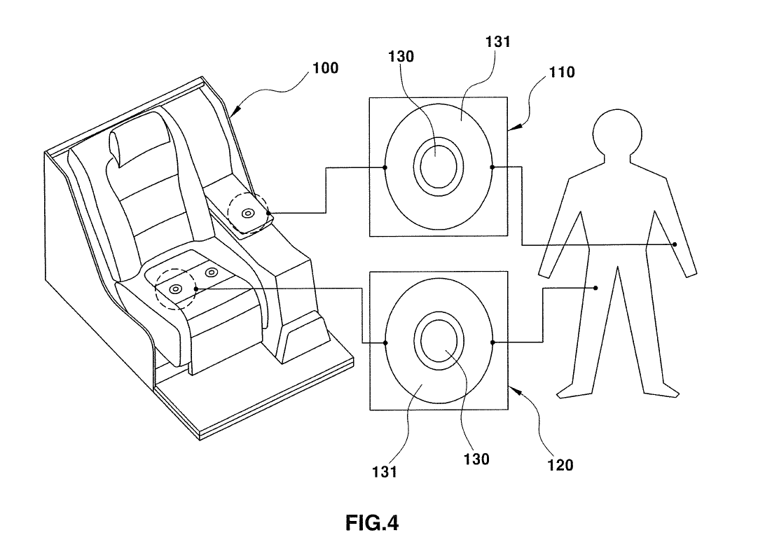

The semi-contact type ECG measurement system of the present inventive concept, as illustrated in FIGS. 1 and 2, includes a contact-type sensor 110 that is disposed in a directly contacting a passenger's skin position (such as an armrest, a door, a seat structure, a handle, a gear lever, etc.) in close proximity to a seat 100 to detect an ECG signal by contacting an exposed skin of the passenger, and a non-contact type sensor 120 which is disposed in a position contacting clothes of the passenger (such as a seat, a backrest, a headrest, etc.) also in close proximity to the seat 100 to detect the ECG signal with a non-contact.

As illustrated in FIGS. 1 and 2, the semi-contact type ECG measurement system includes an ECG sensor module 200 which transmits and processes the ECG signal to provide a stable environment in a vehicle by determining a biological status of the passenger by the ECG signal detected by the contact type sensor 110 and the non-contact type sensor 120.

In the semi-contact type ECG measurement system of the above configuration, the semi-contact type requires a sensor configured by one electrode 130 and a GND 131 detecting an electric signal on a human surface in order to measure the ECG, and in order to detect a good ECG signal, one sensor configured to integrate the electrode 130 and the GND 131 directly contacts the skin and the other sensor detects the ECG with the clothes therebetween by the non-contact type.

In order to determine a state of the passenger by detecting the ECG signal in a vehicle driving state, a good ECG signal may be obtained under various driving conditions such as acceleration, a sudden stop, or imperfect road conditions so as not to cause inconvenience to the passenger.

Further, since the contact-type detection method obtains ECG signals having excellent quality as compared with the non-contact type and the non-contact type has improved convenience because of nonrestraint measurement, the semi-contact type ECG measurement system of the present inventive concept detects a signal at a contact-type level and equals the convenience of the non-contact type.

In this case, the present inventive concept including the contact-type sensor 110 and the non-contact type sensor 120 may further comprise a signal amplifying unit 140 which amplifies a heart-rate signal induced by the electrode 130 to generate the ECG signal.

Furthermore, in the ECG sensor module 200, as illustrated in FIG. 2, a weak ECG signal including noise detected in the electrode 130 provided in each of the contact-type sensor 110 and the non-contact type sensor 120 is primarily amplified by the signal amplifying unit 140, a DC component is removed through a high-pass filter 150, and the output of each sensor 110 or 120 is transferred to the ECG sensor module 200.

Here, the ECG sensor module 200 is configured by a structure which may obtain a good ECG signal by \textcircled{1} removing noise from the ECG signal input from each sensor by a low-pass filter 210, \textcircled{2} amplifying a difference between ECG signals of two sensors and simultaneously removing common mode noise by a differential amplifying unit 220, \textcircled{3} removing a power noise component by passing the signal through a notch filter 230, and \textcircled{4} passing only a component suitable for a ECG frequency in a band pass filter 240.

Finally, Microcontroller 280 of the ECG sensor module 200 is configured by \textcircled{5} converting the good ECG signal into a digital signal in an A/D converter 250 unit, \textcircled{6} detecting a peak through a R-R PEAK detection unit 260, and \textcircled{7} calculating stress through a stress index calculating unit 270 based on accumulated data of the peak.

Accordingly, since various ECG signals are generated according to a health state of the passenger, an attitude, and an environment, the electrode 130 which includes the GND 131 for detecting a differential signal and detects a biological potential directly contacts the skin integrally, and the other electrode 130 detects the ECG signal by measuring the ECG in the clothing state with a non-contact type.

That is, the ECG is measured only by contacting one hand on the electrode 130 while the passenger is comfortably seated, and the stress index of the passenger may be determined by using an index representing various biological states. The Microcontroller feedback may be used to generate a stable state of the passenger, and may be operated in coordination with a service such as ventilation in a vehicle environment.

Furthermore, the ECG signal is detected based on a time axis after contacting and non-contacting (clothing state) a predetermined body part (palms and thighs) through the contact-type sensor 110 and the non-contact type sensor 120 to remove both contact noise and environmental noise through a band pass filter, a notch filter, a GND 131, and a differential amp instead of a DRL circuit.

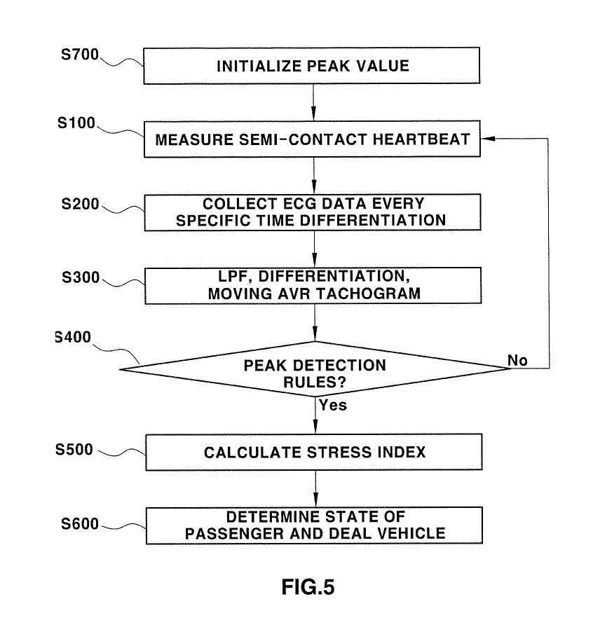

An ECG measurement method will now be described in detail with reference to FIG. 5 accompanied through the above configuration.

An ECG signal of a passenger seated on the seat 100 is detected through the semi-contact type ECG measurement system and then an ECG is measured (S100).

Next, ECG data measured for every predetermined time are stored and collected (S200).

In this case, it is preferred that the predetermined time is set to 0.5 to 1.5 ms so as to measure a detection signal in real time.

ECG R-R PEAK INTERVAL information is obtained in the ECG data collected through filtering (S300).

It is determined whether the obtained ECG R-R PEAK INTERVAL information has regularity (S400).

A stress index is calculated by analyzing and implementing a stress algorithm in the ECG R-R PEAK INTERVAL information through the stress index calculating unit 270 (S500).

A measure and an environment for maintaining stability of the passenger are created through the Microcontroller based on the calculated stress index (S600).

Further, a process (S700) of initializing an ECG signal and a PEAK value collected before the ECG measurement step (S100) is further included.

In addition, by forming the direct-contact type GND integrated electrode 130, a stable signal is ensured by a closed-loop type with the ECG signal in the body and an internal circuit of the ECG sensor, and noise may be removed by bypassing remaining component noise (friction of the clothes, static electricity, external radio wave, the driving noise of the vehicle) except for the noise flowing from the human body through the electrode 130 through the GND 131 to the outside.

Accordingly, as the GND 131 has a larger contact area, a phase of the human signal input to the electrode 130 directly contacting the capacitive-coupling electrode 130 tends to be decreased, and in the differential circuit, the signal quality is improved by removing a large amount of noise of 50 to 60 Hz of a phase signal.

Therefore, when the GND 131 contacts the human body, the ECG signal is measured by a difference between two voltages of two ends of pressure bias resistors of two sensors by forming a closed loop with the ECG signal in the body through a contact of a reference and a body in the two sensors, and when the GND 131 does not contact the human body, the ECG signal is measured by a difference between voltages measured in two sensor units and the ECG is measured by a difference between two signals by measuring a body voltage of one portion of the human body as compared with internal reference of each sensor. As a result, the voltage level of each sensor is more unstable due to changes in internal reference and impedance of the sensor, and thus a DRL method of contacting the body by inverting a predetermined ratio of an average value of measured values of two sensors is used as a security method.

According to the present inventive concept configured above, there are multiple beneficial effects, such as increased convenience by nonrestraint measurement as compared with an existing contact type heart-rate detection method, and excellent ECG signal quality as compared with a non-contact type heart-rate detection method. Further, a body reaction index such as s heart-rate, an ECG, and stress may be calculated with minimal intrusiveness, and a pleasant vehicle environment may be provided by a feedback of an Microcontroller corresponding to the calculated result.

Terms or words used in the present specification and claims, which will be described below should not be interpreted as being limited to typical or dictionary meanings, but should be interpreted as having meanings and concepts which comply with the technical spirit of the present inventive concept, based on the principle that an inventor can appropriately define the concept of the term to describe his/her own inventive concept in the best manner.

Therefore, configurations illustrated in the embodiments and the drawings described in the present specification are only various embodiments of the present inventive concept and do not represent all of the technical spirit of the present inventive concept, and thus it is to be understood that various equivalents and modified examples, which may replace the configurations, are possible when filing the present application.

The inventive concept has been described in detail with reference to exemplary embodiments thereof. However, it will be appreciated by those skilled in the art that changes may be made in these embodiments without departing from the principles and spirit of the inventive concept, the scope of which is defined in the appended claims and their equivalents.

What is claimed is:

1. A semi-contact type ECG measurement system, comprising:
a contact-type sensor positioned to directly contact a skin of a passenger in a vehicle and detect a first ECG signal;
a non-contact type sensor positioned in proximity to the passenger to detect a second ECG signal without directly contacting the skin of the passenger; and
an ECG sensor module configured to process the first and second ECG signals and configured to determine a biological status of the passenger from the first and second ECG signals.

2. The semi-contact type ECG measurement system of claim 1, wherein the ECG sensor module comprises:
a differential amplifying unit configured to amplify the first and second ECG signals and to remove common mode noise;
a band pass filter configured to remove noise;
an A/D converter;
an R-R PEAK detector configured to detect an R-R PEAK INTERVAL; and
a stress index calculator configured to calculate a stress index based on the R-R PEAK INTERVAL.

3. The semi-contact type ECG measurement system of claim 2, wherein the ECG sensor module further comprises a notch filter configured to remove a power noise component from the ECG signal.

4. A semi-contact type ECG measurement method, comprising steps of:
measuring an ECG by detecting an ECG signal of a passenger seated on a seat through a semi-contact type ECG measurement system;
collecting ECG data measured at predetermined times;
obtaining ECG R-R PEAK INTERVAL information in the ECG data;
determining whether the obtained ECG R-R PEAK INTERVAL information has regularity;
calculating a stress index using the ECG R-R PEAK INTERVAL information; and
outputting a measurement Microcontroller based on the stress index.

5. The semi-contact type ECG measurement method of claim 4, further comprising:
initializing the ECG signal and a PEAK value before the step of measuring an ECG.
